(19) United States
(12) Patent Application Publication
(10) Pub. No.: US 2024/0424280 A1
(43) Pub. Date: Dec. 26, 2024

(54) VEIN GRAFT PREPARATION PUMP

(71) Applicant: Quest Medical, Inc., Allen, TX (US)

(72) Inventors: Steve Parnis, Missouri City, TX (US); Andrew Nelson, Dallas, TX (US); Jeffrey Albertsen, McKinney, TX (US); Sukhmani Sandhu, The Colony, TX (US); Adam Hartman, Denton, TX (US)

(21) Appl. No.: 18/749,938

(22) Filed: Jun. 21, 2024

Related U.S. Application Data
(60) Provisional application No. 63/522,554, filed on Jun. 22, 2023.

(51) Int. Cl. A61M 60/135; A61M 60/855

(52) U.S. Cl. CPC A61M 60/135; A61M 60/855

(57) ABSTRACT

The disclosed principles prevent over pressurization of harvested veins by controlling the amount and consistency of pressure applied through the veins from a continuous flow of fluid. This combination of regulated pressurization and continuity/uniformity in the pressure applied through veins is provided using a preparation pump constructed in accordance with the disclosed principles. In some embodiments the pump incorporates a spiral spring to deliver fluids from a bladder within the pump and through a vessel cannula inserted into the grafted vein, and in other embodiments the pump incorporates magnets to compress the bladder. The disclosed apparatus provides only a limited amount of pressure such that no injury is caused to the vein. Pressure and flow are limited and maintained constant, i.e., no pressure changes/spikes, providing uniform pressurization at a predetermined amount safe for the harvested vein, thereby eliminating the human error present with manual pressurization during distension of grafted veins.

### RELATED APPLICATIONS

This disclosure claims priority to U.S. Provisional Patent Application Ser. No. 63/522,554, filed Jun. 22, 2023, which incorporated herein by reference in its entirety for all purposes.

### TECHNICAL FIELD

This disclosure relates generally to the field of vein grafting, and more particularly to a novel vein graft preparation pump for preparing veins for grafting, as well as methods of use and methods of manufacture thereof.

### BACKGROUND

Coronary artery bypass surgery, also called coronary artery bypass grafting (CABG) is a surgical procedure to treat coronary artery disease (CAD), which is, generally speaking, the buildup of plaque in the arteries of the heart. CABG and percutaneous coronary intervention (PCI) are the two methods to revascularize stenotic lesions of the cardiac arteries. PCI is a non-surgical procedure used to treat narrowing of the coronary arteries using a combination of coronary angioplasty with stenting. Conversely, a CABG procedure is a surgical procedure performed to bypass narrowing areas of the heart's arteries by using arteries or veins harvested from other parts of the body. After performing such a bypass, CABG restores adequate blood supply to the heart, thereby slowing the progression of CAD and increasing life expectancy for the patient.

A common CABG procedure involves a saphenous vein graft (SVG) for the bypassing of narrowed areas of the arteries. The great saphenous vein (or long saphenous vein) is a large, subcutaneous, superficial vein of the leg. It is the longest vein in the body, running along the length of the lower limb for returning blood from the foot, leg and thigh to the deep femoral vein. However, long-term patency (i.e., remaining sufficiently open) of SVGs has reported vein graft failure (VGF) is as high as 45% at 18 months after surgery. It is believed that such a high, long-term failure rate is because preparation of SVGs before grafting leads to injury of the vein prior to grafting, which may promote VGF.

Some structural changes of the tunica intima (i.e., the innermost layer) to prevent thickening (i.e., intimal hyperplasia) and vein wall remodeling are necessary for vein graft adaptation to the arterial environment. During and after the harvesting, veins go through a period of ischemia (restriction in blood flow/supply) and reperfusion (tissue damage caused when blood supply returns to tissue after a period of lack of oxygen) after engraftment, which causes damage to endothelial cells (cells that line the interior surface of the vein) and smooth muscle cells (the cells present in the layer surrounding the tunica intima).

Veins are naturally adapted to an environment of low pressure and low blood flow; however, in a graft, veins are transferred and integrated into the arterial circulation, where they are exposed to high pressure and flow. This exposure to arterial pressure and flow causes increased shear stress and wall tension, which further damages the endothelial layer and smooth muscle cells. Over time, such continued damage results in luminal loss that makes the graft more susceptible to atherosclerosis. Progressive atherosclerosis is the primary cause of late vein graft failure.

Conventional preparation of saphenous veins for an SVG typically involves the manual distension (i.e., stretching) of the veins using fluid pressure. For example, U.S. Pat. No. 3,958,557 provides a device and method for preparing a blood vessel, such as the saphenous vein, for use as a coronary bypass graft. FIG. 1 illustrates an isometric view of a cannula 10 constructed in accordance with the principles disclosed in the U.S. Pat. No. 3,958,557. This conventional cannula 10 is comprised of a medical grade polyethylene having the flexibility and softness required to prevent trauma to the intima of the vein in which it is used. The cannula 10 has a hub 14 at the proximal end thereof which is cylindrical and terminates in a planar surface 16. The hub 14 is connected to a body 12 that is diametrically enlarged to form a shoulder 18 in a plane parallel to the surface 16. The hub 14 is sized to accommodate coupling with surgical tubing (not shown), which is press-fit on the hub 14 and abuts shoulder 18. The body 12 is frustoconical in configuration, the forwardly tapering ramp surface 20 merging with stylet 22, which forms the distal end of the cannula 10. The stylet 22 has a very gradual taper from the body 12 to the distal end 24. The external dimension of the stylet 22 is selected to be essentially the normal internal diameter of a suitable vein segment for use in coronary bypass. The stylet 22 includes a peripheral flange 26 spaced slightly behind the distal end 24. The peripheral flange 26 has a forwardly tapering ramp surface 28 that facilitates insertion of the stylet 22 into a suitable vein segment 30. The flange 26 terminates in a shoulder 32 which permits pressure-tight ligation, as described below.

FIG. 2 illustrates a side, partial cross-section view of the conventional cannula 10 taught in U.S. Pat. No. 3,958,557. The stylet 22 has an annular bore 34 that opens at the distal end 24 and is also in open communication with hollow 38 in the hub 14. The hollow 38 is coaxial with the bore 34 and is provided with a Luer taper so as to form a female coupling. Thus, the cannula 10 is press-coupled to an irrigation instrument such as a syringe 40. The selected portion of the vein 30 is then severed and the stylet 22 inserted within the lumen of the resected vein 30 until the vein 30 is pressed tightly upon the ramp surface 20. Thereafter, temporary ligation is effected by tying suture 42 tightly around the vein 30 near the flange 26. Ligation will have the effect of creating a fluid seal between the vein segment 30 and the cannula 10.

Thereafter, a syringe 40 is used for irrigating purposes to determine whether occlusions or clots remain in the vein segment 30 and, if so, to accommodate flushing of the vein 30. A distant portion of the vein segment 30 can be clamped and manual hydrostatic pressure communicated to the vein through the cannula 10 with the syringe 40. This applied pressure is used to detect leaks in the vein segment 30, particularly at the severed tributary sites. This pressure also serves to determine the distensibility index of the vein 30. If the vein 30 distends too easily under pressure, it is not suitable for the coronary artery bypass. Assuming the vein 30 has been proven under this manual pressurization, the vein 30 is used for the coronary artery bypass.

However, data has shown that acute damage to the vein graft intima can result from extensive distension of such veins during repair of the severed tributaries of the vein graft, but that this does not necessarily occur at pressure equivalent to normal arterial pressure. However, manual pressure distension does not limit or otherwise regulate the pressure of fluid applied and does not circulate the fluid during preparation. Consequently, intimal damage, although unknown to the clinician at the time, is often caused to the grafted vein when prepared using such manual procedures due to high pressurization as a result of unregulated manual pressurization techniques found in U.S. Pat. No. 3,958,557 as well as other conventional approaches. Accordingly, what is needed in the art is a device and related method for limiting the pressure of the vein graft during preparation for a vein graft to be used in a coronary artery bypass procedure, that does not suffer from the deficiencies found with conventional vein preparation approaches. The disclosed principles provide such a unique device, methods of use, and methods of manufacture thereof.

### SUMMARY

The disclosed principles prevent over pressurization of veins harvested for vein grafts by controlling the amount and the consistency of pressure applied through the harvested veins from a continuous flow of fluid through the vein graft. This combination of regulated pressurization and continuity/uniformity in the pressure applied through harvested veins is provided using a unique vein graft preparation pump constructed in accordance with the disclosed principles. Generally speaking, the apparatus incorporates a spiral spring with a predetermined spring constant (K) that is sufficient to deliver fluids from a bladder within the pump and through a vessel cannula inserted into the vein section selected for the grafting procedure. Additionally, the disclosed apparatus provides only a limited amount of pressure such that no injury is caused to the vein. Pressure and flow are limited and maintained constant via use of the spiral spring, i.e., no pressure changes/spikes, which provides uniform pressurization at a predetermined amount safe for the harvested vein section, thereby eliminating the human error present with manual pressurization during distension of grafted veins.

Additional embodiments and advantages and variation thereof are also encompassed within the scope of the disclosed principles, and some such exemplary embodiments discussed in further detail herein.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawing, in which:

FIG. 1 illustrates an isometric view of a cannula constructed in accordance with the prior art, and used in the irrigation of a vein harvested for a grafting procedure;

FIG. 2 illustrates a side, partial cross-section view of the conventional cannula illustrated in FIG. 1;

FIG. 3 illustrates a front isometric view of one embodiment of a vein graft preparation pump designed and constructed in accordance with the disclosed principles;

FIG. 4 illustrates an exploded isometric view of the vein graft preparation pump illustrated in FIG. 3;

FIG. 5 illustrates an isometric view of the drive system of the embodiment of a vein graft preparation pump illustrated in FIGS. 3 and 4;

FIG. 6 illustrates one embodiment of a pump assembly, having the pump illustrated in FIGS. 3 and 4, ready for use in a vein irrigation procedure;

FIG. 7A illustrates a front isometric view of an alternative embodiment of a vein graft preparation pump designed and constructed in accordance with the disclosed principles;

FIG. 7B illustrates a front isometric view of the vein graft preparation pump of FIG. 7A, but in a “Flow” position as compared to the “Stop” position shown in FIG. 7A;

FIG. 8 illustrates a profile, partially transparent view of the vein grafting preparation pump illustrated in FIGS. 7A and 7B; and

FIG. 9 illustrates an exploded isometric view of the vein graft preparation pump illustrated in FIG. 8.

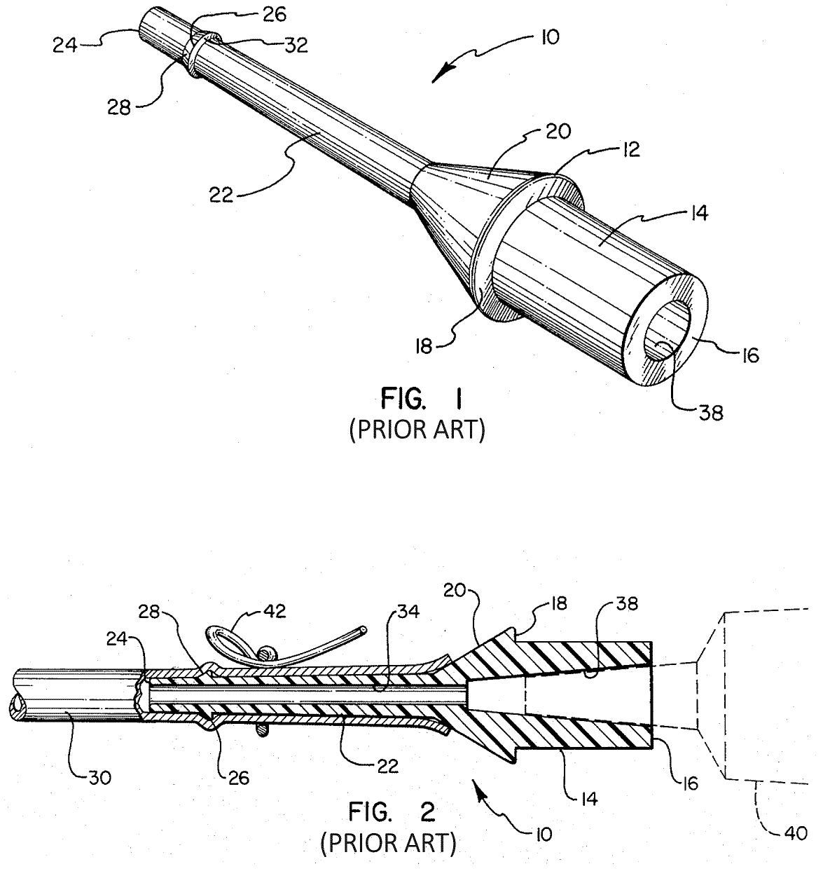

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Although multiple embodiments are shown and discussed in great detail, it will be apparent to those skilled in the relevant art that some features that are not relevant to the present invention may not be shown for the sake of clarity.

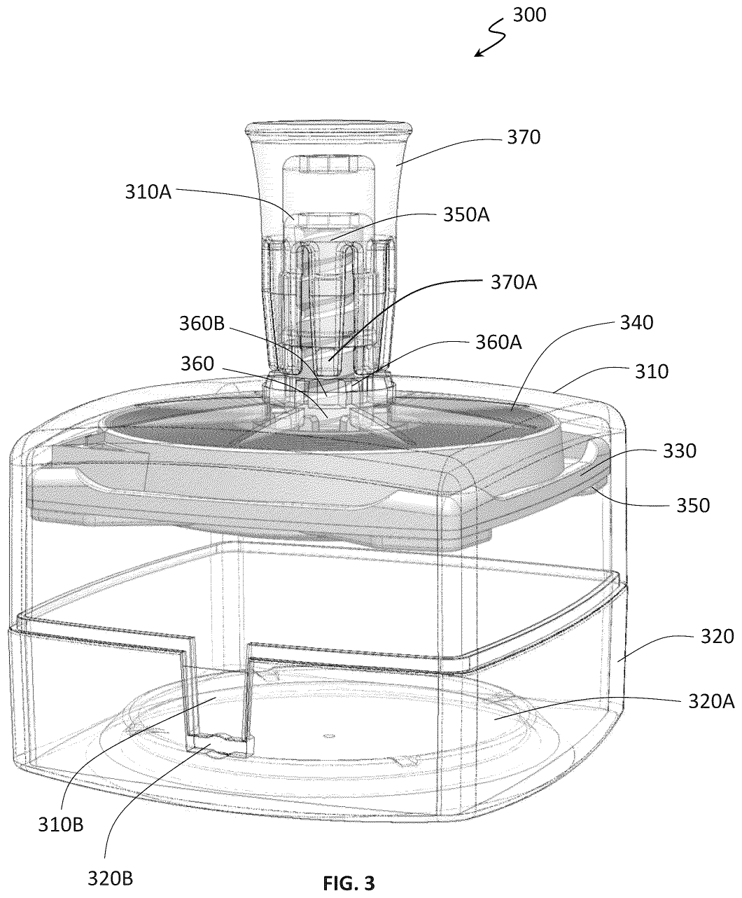

Looking now at FIG. 3, illustrated is a front isometric view of one embodiment of a vein graft preparation pump 300 designed and constructed in accordance with the disclosed principles. As mentioned above, the vein graft preparation pump 300 provides a combination of regulated pressurization and consistency in the pressure applied via a fluid pushed through harvested veins that are used in bypass procedures. This uniform pressurization at a predetermined amount safe for the harvested vein section (typically a range of about 2-4 psi, and in particular 2.7 psi) eliminates the human error that too often causes over pressurization due to manual pressurization during irrigation/distension of grafted veins. This view of the pump 300 is shown as partially transparent in order to better illustrate the cooperation of some of the components therein.

The components of the illustrated embodiment of the vein graft preparation pump 300 include a housing comprised of an upper case 310 and a lower case 320 connected together and housing the various pump components therein. In a seat in the lower case 320 is a fluid bladder 320A that holds fluid, for example, saline, used to irrigate a harvested vein section during the distension process. In exemplary embodiments, the bladder 320A is a 50 ml bladder; however, any size bladder may be employed with a preparation pump 300 as disclosed herein. Exiting from a port 320B in the lower case 320 would be a hose or other fluid conduit (not illustrated) having its proximal end connected to the bladder 320A, and its distal end connected to a cannula (not illustrated) onto which is sutured a grafted vein prior to the irrigation procedure.

To expel the fluid from the bladder 320A, a platen 350 is moved downwardly via a drive system (discuss in detail below) to compress the bladder 320A in order to expel the fluid to irrigate the harvested vein (not illustrated). As the platen 350 is compressed onto the bladder 320A by the drive system of the illustrated pump 300, fluid is forced from the bladder 320A and out through the cannula and into the vein. As discussed in further detail below, the downward movement of the platen 350 to compress the bladder 320A is provided by the pump 300 at a steady rate, which in turn provides a consistent and maximum pressure of fluid through the grafted vein during use of the pump 300.

The drive system driving the platen 350 is comprised of a spring seat 330 holding a spring 340. The spring 340 in this embodiment is a clock spring or spiral spring, and is configured to turn a drive nut 360 having internal threads corresponding to external threads formed on a shaft 350A of the platen 350. Specifically, the spring 340, which is connected at one end to the spring seat 330 and at its other end to the drive nut 360, unwinds such that it spins the drive nut 360 around the platen shaft 350A. With the spring seat 330 and drive nut 330 locking in position within the upper case 310, the rotation of the drive nut 360 about the platen shaft 350A causes the platen shaft 350A to move downwardly through the drive nut 360. As the platen shaft 350A moves downwardly through the drive nut 360, the platen 350 correspondingly moves downwardly as well to compress the bladder 320A within the lower case 320.

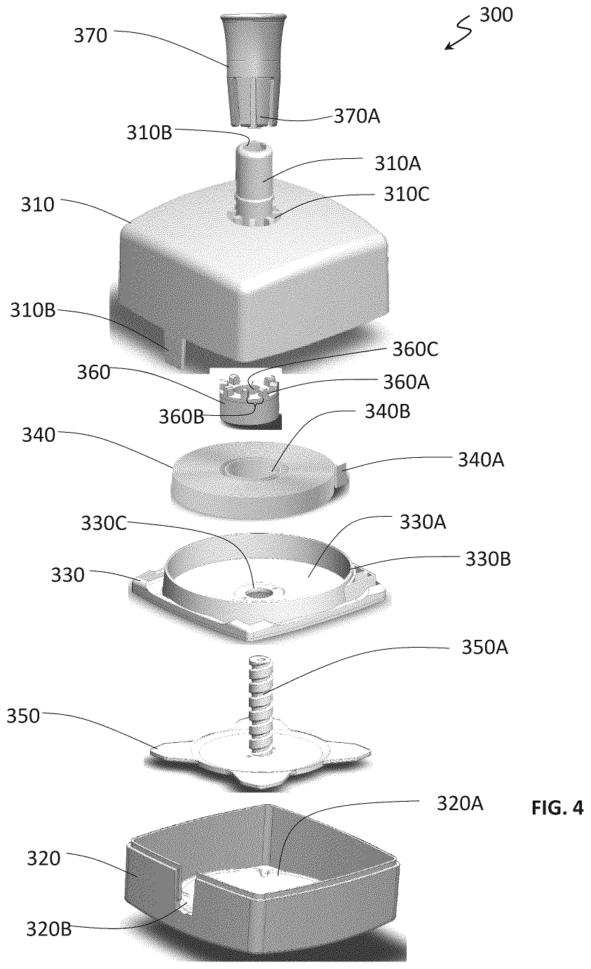

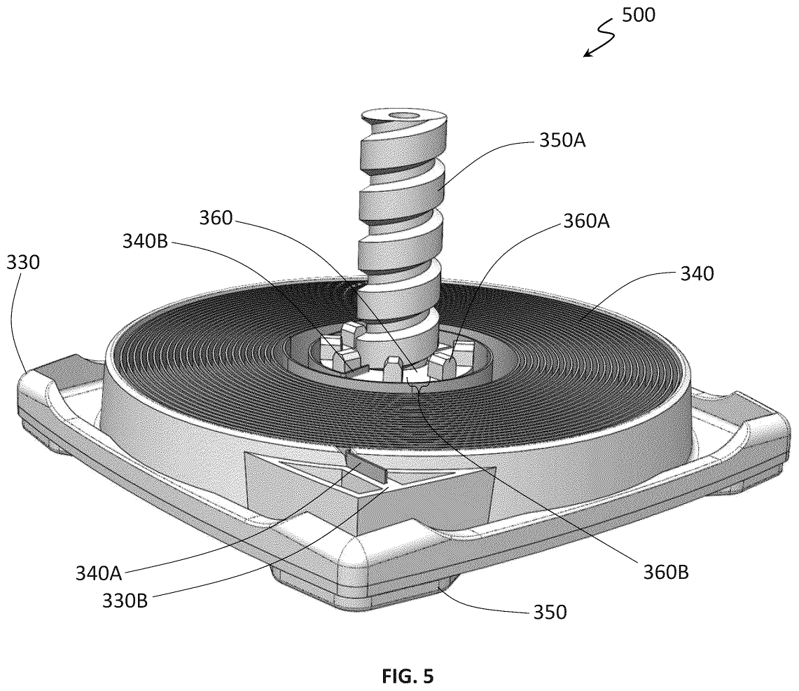

The pump 300 also includes a lock 370 that is slidably engaged over a post 310A extending from the top of the upper case 310. The lock 370, which is slidable upward and downward along the upper case post 310A, includes a plurality of fingers 370A at its lower end. These fingers 370A are configured to slide through corresponding holes 310C (see FIG. 4) formed in the top of the upper case 310 in order to engage corresponding slots 360B formed between teeth 360A on the top of the drive nut 360. As a result, when the lock 370 is slid down the post 310A such that the fingers 370A engage the slots 360B on the top of the drive nut 360, the drive nut 360 is prevented from turning even under power of the spring 340. Conversely, when the lock 370 is slid up the post 310A such that the fingers 370A disengage from the slots 360B on the top of the drive nut 360, the drive nut 360 is permitted to turn via the power of the spring 340, which in turn lowers the platen 350 such that it compresses the bladder 320A.

Turning now to FIG. 4, illustrated is an exploded isometric view of the vein graft preparation pump 300 illustrated in FIG. 3. This exploded view not only provides more detailed views of each component of the pump 300, but also illustrates how each of these components are assembled to form the pump 300. In particular, to assemble the pump 300, the spring 340 is seated in the spring seat 330. A first end 340A of the spring 340 is secured in place via a first tab lock 330B formed in the spring seat 330. The second end 340B of the spring 340 is secured to the drive nut 360 via a second tab lock formed in the drive nut (see FIG. 5).

The shaft 350A of the platen 350 is then passed through a central hole 330C in the spring seat 330, through the middle of the spring 340, and threaded into the drive nut 360. The platen shaft 350A is located into a central opening 310B of the post 310A extending from the upper case 310. The bladder 320A is placed within the lower case 320, and may either be filled with whatever fluid is desired (e.g., saline), or the bladder 320A may be filled after assembly of the pump 300, such as immediately prior to its use in an irrigation procedure. The upper case 310 and the lower case 320 are then connected together, which may be an openable connection or a permanent connection. In some embodiments, the cases are snap fit or press fit together, while in other embodiments, the cases are glued together or otherwise permanently bonded together, such as be a welding process. In exemplary embodiments, the cases are formed from plastic, and in some embodiments the two may be secured together via a plastic welding process. An alignment tab 310B of the upper case 310 is sized and configured to fit with a complimentary slot 320B.

Once the upper case 310 and lower case 320 are joined with the spring 340, spring seat 330, platen 350, and drive nut 360 held within, the lock 370 may then be slid over the post 310A. In advantageous embodiments, the lock 370 may be snap fit to the post 310A such that when it is raised and lowered to permit and stop rotation of the drive nut 360, it does not come lose from the post 310A. As discussed above, when the lock 370 is pushed downward to prevent turning of the drive nut 360 via the spring 340, the fingers 370A engage corresponding slots 360B in the drive nut. Then, when operation of the pump 300 is desired again, the lock 370 may be lifted upward until the fingers 370A disengage from the slots 360B, thereby permitting the spring 340 to rotate the drive nut 360 about a central axis defined through the platen shaft 350A. With the drive nut 360 secured in place within the upper case 310, such as by coupling the spring seat 330 to an underside of the upper case 310, the rotation of the drive nut 360 forces the platen shaft 350A to translate downward along the central axis. As the platen shaft 350A translates downwardly through the drive nut 360, the flat portion of the platen 350 compresses the bladder 320A held in the lower case 320.

Looking now at FIG. 5, illustrated is an isometric view of an exemplary drive system 500 for the vein grafting preparation pump 300 illustrated in FIGS. 3 and 4. This embodiment of a drive system 500 includes the spring seat 330 into which the spring 340 is held. The first end 340A of the spring 340 is secured in place via a first tab lock 330B formed in the spring seat 330. The second end 340B of the spring 340 is secured to a similar locking slot formed in the drive nut 360. With the second end 340B of the spring 340 secured to the drive nut 360, the spring 340 will rotate the drive nut 360 about a central axis of the spring 340.

As shown, the shaft 350A of the platen 350 is positioned through the center of the spring 340 via a hole formed in the spring seat 330. The exterior of the shaft 350A and the interior of the drive nut 360 are formed with complimentary threads such that rotation of the drive nut 360 by the spring 340 causes the shaft 350A to move vertically through the spring seat 330. Although the threads in the drive nut 360 and shaft 350A are illustrated as square threads, any type or shape of threads may be provided. As the spring 340 rotates the drive nut 360 during use of the pump 300, the shaft 350A moves downwardly through the spring seat 330. This downward movement of the shaft 350A causes the platen 350 to move downward within the upper and lower case 310, 320 of the pump 300. The platen 350 is sized and shaped corresponding to the interior of the upper and lower case 310, 320 such that it cannot rotate with the case during use of the pump 300. Thus, since the platen 350 and its shaft 350A cannot rotate with respect to the case, the two move downwardly through the spring seat 330. As the platen 350 continues to move downward, it contacts and compresses the bladder containing a fluid to be used in an irrigation or other fluid-based process.

As noted above, the pump 300 may also be stopped and started during its use. Specifically, the drive nut 360 includes teeth 360A formed on the top of the drive nut 360, and corresponding slots 360B formed between the teeth 360A. The lock 370 discussed above includes fingers 370A that are sized and shaped to engage the slots 360B. When so engaged in the slots 360B, the drive nut 360 and lock 370 would rotate together; however, as also discussed above, the fingers 370A initially pass through corresponding holes 310C formed in the upper case 310 immediately above the teeth 360A and slots 360B on the drive nut 360. Therefore, these holes 310C will hold the fingers 370A in place and keep the lock 370 from rotating, which in turn prevents the drive nut 360 from rotating when the fingers 370A are engaged in the slots 360B. Conversely, to permit the drive nut 360 to rotate during use of the pump 300, the lock 370 is moved upward such that the fingers 370A no longer engage the slots 360B on the drive nut 360. Once the fingers 370A are out of the slots 360B, the drive nut 360 is permitted to rotate under power of the spring 340.

To wind the spring 340 within the pump 300 such that the pump 300 is ready for use, the spring 340 may be wound during manufacture of drive system 500 of the pump 300. In such embodiments, the platen 350 is already positioned at the top of the interior of upper case 310. Therefore, the bladder could be filled with a desirable fluid, such as saline, at the time of manufacture. As such, the pump 300 could be completely ready to use once removed from its packaging. Alternatively, the bladder could be filled while the user is preparing to use the pump 300 in a desired procedure. In other embodiments, the spring 340 may be wound after the pump 300 is manufactured. Specifically, the pump 300 may be constructed with the platen 350 in its lowest position, i.e., having fully compressed the bladder. Then, prior to use, the user may move the platen 350 into its highest position up inside the upper case 310. As the platen 350 is moved upward manually, the threads of the shaft 350A will cause the drive nut 360 to rotate. This rotation of the drive nut 360 is in the opposite direction to its rotation during use of the pump 300, and thus this opposite rotation will wind the spring 340. Once the platen shaft 350A is moved all the way through the drive nut 360, and thus the platen 350 is positioned up inside the upper case 310, giving plenty of room for the bladder to be filled with the desired fluid.

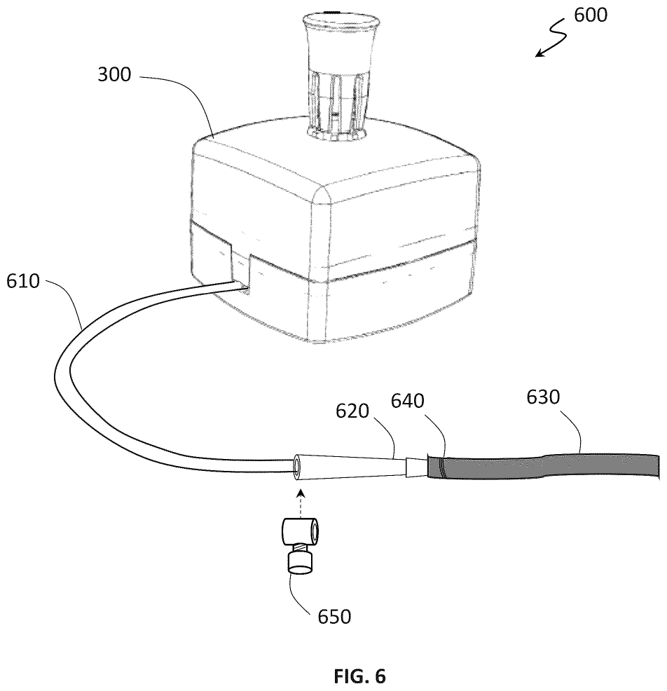

Turning finally to FIG. 6, illustrated is a pump assembly 600, having the pump 300 illustrated in FIGS. 3 and 4, ready for use in a vein irrigation procedure. Specifically, the pump assembly 600 includes the pump 300, as well as tubing 610 connected to the bladder (not illustrated) within the pump 300, and a cannula 620 attached to the distal end of the tubing 610.

The tubing 610 may be comprised of any advantageous material, such as medical grade surgical tubing manufactured for use in various medical procedures. Moreover, the tubing 610 may be permanently connected to the bladder, or may be removably connected such that the tubing is affixed to the bladder prior to use of the pump 300. Similarly, the cannula 620 may be permanently connected to the tubing 610, or may be removably connected such that the cannula 620 is connected to the tubing 620 prior to use of the pump 300. Moreover, the cannula 620 may be constructed of any advantageous material, such as a medical grade plastic suitable for use in medical procedures.

A vein section 630 is also illustrated, which is to be prepared for grafting such as in a bypass procedure. To prepare the vein 630 for such a procedure, the vein is irrigated in a distension procedure in the manner discussed above. To employ a pump 300 in accordance with the disclosed principles for such a procedure, the vein 630 is connected to the cannula 620. For example, the vein 630 is typically held on the cannula 620 by sutures 640, but other means of holding the vein 630 on the cannula 620 may also be employed. In some embodiments, the vein 630 may be sutured onto the cannula 620 prior to the connecting of the cannula 620 to the tubing 610. For the irrigation procedure, a fluid, such as saline, is stored in the bladder of the pump 300. As mentioned above, the fluid may be provided in the bladder at the time of manufacturing the pump 300, and in other embodiments the fluid is injected into the bladder prior to use of the pump 300. For example, a syringe or other device may be used to inject a predetermined amount of fluid, such as 50 ml, into the bladder immediately prior to use in the vein 630.

Once the vein 630 is attached to the cannula 620 and the fluid is held in the bladder, the pump 300 may be operated to pump the fluid into and through the vein 630. To do so, the lock 370 is lifted upward by a user, which removes the fingers 370A from the locking slots 360A in the drive nut 360. Once the lock 370 is so lifted, the spring 340, which was either wound at the time of manufacture of the pump 300 or wound by a user prior to use of the pump 300, rotates the drive nut 360 in the manner described above. The rotation of the drive nut 360 moves the platen shaft 350A is moved downward through the drive nut 360. This causes the platen 350 to move downward and compress the bladder holding the fluid. This compression forces the fluid out of the bladder, through the tubing 610 and cannula 620, and into the vein 630. In accordance with the disclosed principles, the pump 300 may be permitted to operate and dispense the fluid continuously, or it may be stopped in the manner discussed above by vertical movement of the lock 370.

The disclosed pump 300 provides only a limited amount of pressure such that no injury is caused to the vein. Specifically, the spiral construction of the spring 340 provides a steady and substantially uniform rate of rotation of the drive nut 360, and thus the substantially uniform downward movement of the platen 350 to compress the bladder in a correspondingly uniform manner. Pressure is limited and maintained constant via use of the spiral spring 340 and the components moved by the spiral spring 340, which provide uniform pressurization at a predetermined amount safe for the harvested vein section 630, i.e., typically in the range of 2-4 psi, and preferably about 2.7 psi. Thus, the spring 340 is selected so that only a specific amount of pressure, or a range of pressures, of the fluid expelled from the bladder is provided by the pump 300, based on the rate of rotation of the drive nut 360. The faster the rate of rotation of the drive nut 360, the faster the platen 350 compresses the bladder, and thus the higher the pressure that the fluid is expelled from the pump 300. Conversely, the slower the rate of rotation of the drive nut 360, the slower the platen 350 compresses the bladder, and thus the lower the pressure that the fluid is expelled from the pump 300. The tension of the spring 340 may be selected at the time of manufacturing to adjust the amount of substantially uniform pressure being provided from the pump 300, which will vary based on the desired use of the pump 300. Additionally, the tension of the spring is predetermined based on the size of the inner diameter of the tubing 610 and the cannular 620, as well as taking into consideration the density of the fluid to be used with the pump 300. This combination of regulated pressurization and continuity in the pressure applied through harvested veins 630 results in a constant, safe pressure applied from a vein graft preparation pump as disclosed herein to the harvested veins 630. Moreover, this uniform and regulated pressure provided by a pump as disclosed herein eliminates the human error present with manual pressurization during distension of grafted veins.

Furthermore, in some embodiments of pump assembly 600 having a preparation pump 300 in accordance with the disclosed principles, a fail-safe pressure release valve 650 may be included. Such a pressure release valve 650 may be used to ensure a maximum pressure (which may be determined by the type/size of harvested vein 630, the type/viscosity of the fluid be used, or any other factor or combination of factors) is not exceeded when employing the pump 300 to prepare the harvested vein 630. Such a pressure release valve 650 may take the form of valve located at the junction between the tubing 610 and cannula 620, or manufactured in the cannula 620 itself, which will automatically open to release pressure at a predetermined pressure amount. For example, in one embodiment, such a pressure relief valve 650 may be configured to open if the pressure exceeds about 4 psi. In a more specific embodiment, such a pressure relief valve 650 may be configured to open if the pressure exceeds about 2.7 psi. Alternatively, the valve 650 may instead be provided as a pressure regulator rather than a relief valve. In such embodiments, the pressure regulator 650 will permit flow through the vein 630 at only the desired pressure (e.g., about 2.7 psi) or at a desired pressure range (e.g., 2-4 psi). In yet other embodiments, a combination of pressure relief valve and a pressure regulator may be employed, which can be beneficial in situations where the regulator fails at the desired pressure regulation, and thus the pressure relief valve provides a safety redundancy in preventing over-pressurization of a vein during its irrigation.

Additionally or alternatively, a damper (not illustrated) may also be provided within the pump 300. An exemplary damper may employ a piston and accompanying fluid selected so that only a maximum amount of pressure can be applied with the movement of the platen 350 depressing the bladder during use of the pump 300. A damper may also assist in ensuring that a consistent and uniform amount of pressure is provided by the pump 300, where spring 340 alone may not provide the same amount of pressure at the beginning of the unwinding of the spring 340 than is applied at the end of the unwinding of the spring 340. Also, such a damper could include an adjustment mechanism, which would permit a user of a pump 300 as disclosed herein to adjust the amount of pressure in the fluid being expelled from the pump 300 during use, while also ensuring that amount of pressure does not exceed a maximum and is provided uniformly during use. Moreover, such a damper may be positioned in any advantageous location within the pump 300 without departing from the scope of the principles disclosed herein. Furthermore, a damper may be employed with the spring 340, which can further regulate the unwinding of the spring 340 such that substantially uniform, and a maximum amount of, pressure is expelled from the pump 300.

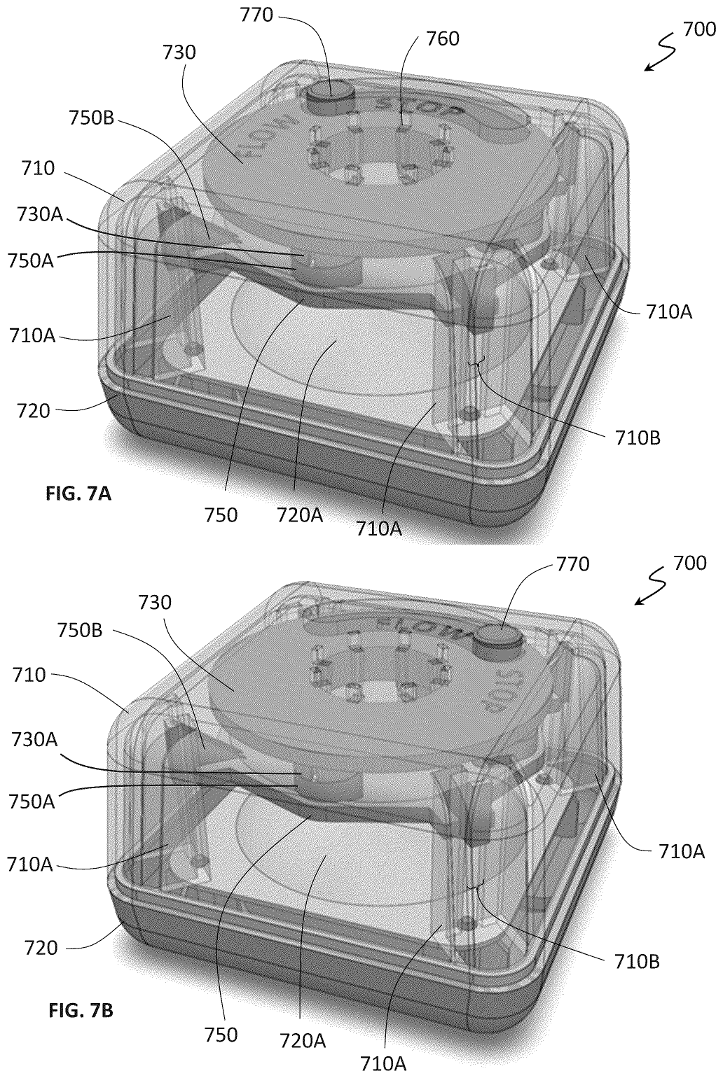

Turning now to FIGS. 7A-7B, FIG. 7A illustrates a front isometric view of an alternative embodiment of a vein graft preparation pump 700 designed and constructed in accordance with the disclosed principles. FIG. 7B illustrates a front isometric view of the vein graft preparation pump 700 of FIG. 7A, but in a “Flow” position as compared to the “Stop” position shown in FIG. 7A. As with the pump embodiments discussed above, the vein graft preparation pump 700 provides a combination of regulated pressurization and consistency in the pressure applied via a fluid pushed through harvested veins that are used in bypass procedures. These views of the pump 700 are shown as partially transparent in order to better illustrate the cooperation of some of the components therein.

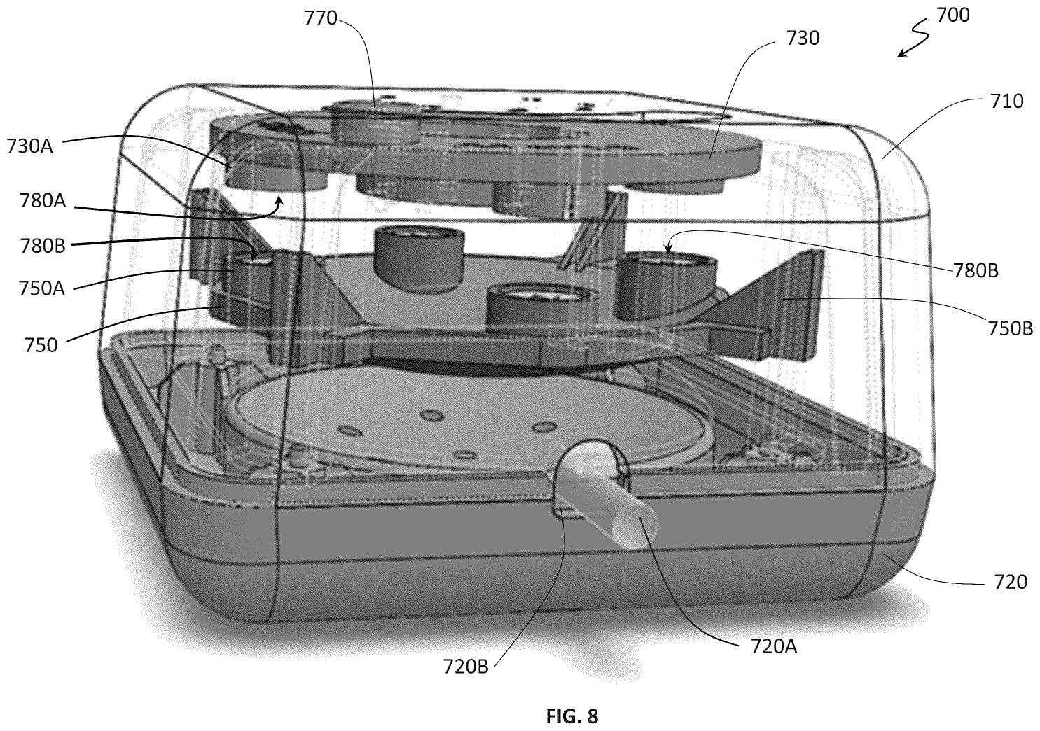

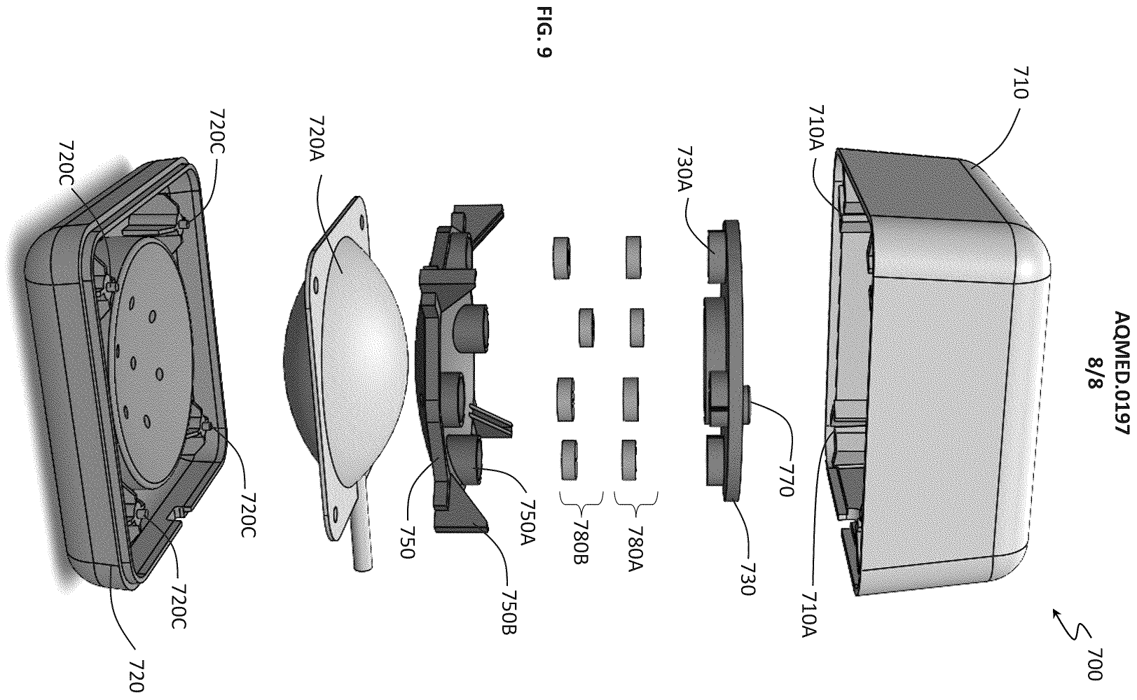

The components of this embodiment of the vein graft preparation pump 700 again include a housing comprised of an upper case 710 and a lower case 720 connected together and housing the various pump components therein. In a seat in the lower case 720 is a fluid bladder 720A that holds fluid, for example, saline, used to irrigate a harvested vein section during the distension process. In exemplary embodiments, the bladder 720A is a 50 ml bladder; however, any size bladder may be employed with a preparation pump 700 as disclosed herein. Exiting from a port 720B (see FIG. 8) in the lower case 720 is a hose or other fluid conduit having its proximal end connected to the bladder 720A, and its distal end connected to a cannula (not illustrated) onto which is sutured a grafted vein prior to the irrigation procedure.

To expel the fluid from the bladder 720A, a platen 750 is moved downwardly via a drive system (discuss in detail below) to compress the bladder 720A, similar to the embodiments discussed above, to expel the fluid to irrigate the harvested vein (not illustrated). As the platen 750 is compressed onto the bladder 720A by the drive system of pump 700, fluid is forced from the bladder 720A and out through the cannula and into the vein mentioned above. As discussed in further detail below, the downward movement of the platen 750 to compress the bladder 720A is provided by the pump 700 at a steady rate, which in turn provides a consistent and maximum pressure of fluid through the grafted vein during use of the pump 700.

The drive system driving the platen 750 is comprised of opposing magnetic forces provided by magnets (see FIG. 8) positioned within magnet seats 730A formed at the bottom of a moveable seat 730. Corresponding opposing magnets (see FIG. 8) are positioned within magnet seats 750A formed on the top of the platen 750. The polarities of the magnets in the seats 730A and platen seats 750A are aligned such that the same polarity of the upper and lower magnets face each other (i.e., the positive polarity of the upper and lower magnets face each other, or the negative polarities face each other).

A central spool 760 is provided extending from the upper, interior surface of the upper case 710, about which the seat 730 is configured to rotate. Specifically, the seat 730 comprises a central aperture that is sized to receive the spool 760 therethrough with a slip fit engagement. A handle 770 is provided extending upwardly from the seat 730 and extending through the top surface of the upper case 710 so that it may be manipulated by a user of the pump 700 (i.e., switched between “Stop” and “Flow” positions). Before initial use of the pump 700, the seat 730 is rotated in its “Stop” position (as illustrated in FIG. 7A) with respect to the platen 750 such that the magnets in upper seats 730A and magnets in platen seats 750A are not vertically aligned. This lack of vertical alignment between the sets of upper and lower magnets results in no opposing magnet forces acting between the seat 730 and the platen 750. Moreover, the fluid in the bladder 720A would be full before initial use of the pump 700 (either full and sealed at manufacture, or filled by a user through injection of the fluid into the bladder 720A), and thus the fully inflated bladder 720A would provide an upward force on the platen 750 to keep it in its upper position since there is no opposing magnetic force being applied between the seat 730 and the platen 750 due to their respective magnets being misaligned while the seat 730 is in the “Stop” position.

When the handle 770 is rotated to a “Flow” position (as illustrated in FIG. 7B), the seat 730 is rotated a distance sufficient to vertically align the magnets within the upper seats 730A and the platen seats 750A. Then, because the same magnetic polarities for these upper and lower magnets are facing each other, the opposing magnetic force causes the platen 750 to begin moving downward within the upper case 710. In alternative embodiments, pairs of upper and lower magnets having opposing fields are provided along with pairs of upper and lower magnets having attractive fields in the seat 730 and platen 750. In such embodiments, when the handle 770 rotates the seat 730 to the “Flow” position, the pairs of magnets having opposing forces are aligned, and thus provide the downward force to the platen 750. When the handle 770 rotates the seat 730 to the “Stop” position, the pairs of magnets having attractive forces may be aligned, while the pairs of magnets with the opposing fields are no longer aligned, and thus provide the upward force to remove any pressure by the platen 750 onto the bladder 720A or even cause the platen 750 to move upwardly within the upper case 710 in some embodiments.

Fins 750B formed at each of the corners of the platen 750 are sized and configured to be received (e.g., via slip fit) and thus slide within corresponding vertical channels 710B formed at the corners within the interior of the upper case 710. The alignment of the fins 750B and the channels 710B permit the vertical movement of the platen 750 from its upper to its lower position within the upper case 710, but without permitting the platen 750 to rotate. This maintained nonrotational alignment keeps the alignment of the opposing magnets in the seat 730 and the platen 750 such that the opposing magnetic fields move the platen 750 downward. The strength of the magnetic fields of the upper and lower magnets should be selected so as to be strong enough to compress the platen 750 against the bladder 720A as the platen 750 is moved downwardly. Additionally, the strength of the magnets is selected to provide a desired flow rate of the fluid being expelled from the bladder 720A via its compression by the platen 750. The selected strength will vary based on the viscosity of the fluid, the size of the output fluid conduit from the bladder 720A, and the desired flow rate of the expelling fluid, and accounting for any friction between the fins 750B and the vertical channels 710B. For example, if saline is the fluid within the bladder 720A and the desired flow rate from the pump 700 is 2-4 psi, then the amount of magnetic fields/opposing forces of the magnets is selected based on the size of the output from the pump 700 for the viscosity of saline and the desired output flow rate/pressure. Those skilled in the art will easily be able to calculate a desired strength of magnetic field based on the specific parameters of each's desired use of the pump 700.

Looking now at FIG. 8, illustrated is a profile, partially transparent view of the vein grafting preparation pump 700 illustrated in FIGS. 7A and 7B. This view illustrates the “Flow” alignment of the drive system of this embodiment of a pump in accordance with the disclosed principles. Specifically, the seat 730 and platen 750, as well as their respective magnet seats 730A, 750A may be seen. The upper magnets 780A are shown being secured within the magnet seats 730A, and the lower magnets 780B are shown secured within the magnet seats 750A.

As shown, as the upper and lower magnets 780A, 780B are aligned during use of the pump 700, the opposing magnetic forces push the platen 750 downwardly. This downward movement of the platen 750 within the upper and lower case 710, 720 of the pump 700 compresses the bladder 720A and thereby expels the fluid out of the bladder, through a fluid conduit and cannula, and into the vein being prepared for graft procedure. As discussed above, the platen 350 includes fins 750B that slide within corresponding vertical channels 710B formed on the interior of the upper case 710 such that the platen 750 cannot rotate with the case 710 during use of the pump 300.

As noted above, the pump 700 may also be stopped and started during its use. Specifically, the seat 730 includes a handle 770 usable by a user to partially rotate the seat 730, about the central spool 760, with respect to the platen 750. This rotation aligns or misaligns the upper and lower magnets 780A, 780B to provide or remove, respectively, the downward force on the platen 750. The ability to stop the fluid expelling from the pump 700 during irrigation/distension of a vein graft permits any detected fluid leaks in the vein to be sutured, and thereafter continue the fluid flow through the vein to check for any additional leaks, etc. To reset the pump 700 for an additional or further irrigation process, a user may simply switch the handle 770 to the “Stop” and then inject additional fluid into the bladder 720A. With the pump 700 then ready for use, the handle 770 may again be moved to the “Flow” position to once again align the magnets 780A, 780B to expel the newly refilled fluid from the pump 700.

Turning finally to FIG. 9, illustrated is an exploded isometric view of the vein graft preparation pump 700 illustrated in FIG. 8. This exploded view provides a more detailed view of each component of the drive system within the pump 700. In particular, the drive system includes the seat 730 which includes magnet seats 730A holding the upper magnets 780A, as well as the platen 750 that includes magnet seats 750A holding the lower magnets 780B. In addition to the drive system, the fluid bladder 720A, which is compressed by the drive system, is also visible.

The bladder 720A is placed within the lower case 720, and may either be filled with whatever fluid is desired (e.g., saline), or the bladder 720A may be filled after assembly of the pump 700, such as immediately prior to its use in an irrigation procedure. The upper case 710 and the lower case 720 are then connected together, which may be an openable connection or a permanent connection. In some embodiments, the cases 710, 720 are snap fit or press fit together, while in other embodiments, the cases are glued together or otherwise permanently bonded together, such as be a welding process. In exemplary embodiments, the cases 710, 720 are formed from plastic, and in some embodiments the two may be secured together via a plastic welding process. Alignment tabs 720C of the lower case 720 are sized and configured to fit with complimentary slots 710A formed in the interior of the upper case 710. These tabs and slots 720C, 710A may also be sized to provide the connective force keeping the upper and lower cases 710, 720 together. In embodiments where an adhesive is used to keep the upper and lower cases 710, 720 together, the adhesive may be applied at the points where the tabs and slot connect.

While this disclosure has been particularly shown and described with reference to preferred embodiments, it will be understood by those skilled in the pertinent field art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

The uses of the terms “a” and “an” and “the” and similar references in the context of describing the invention(s) (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. A pump providing a consistent regulated pressurization of an output fluid, the pump comprising:
a housing containing a bladder configured to store a fluid therein and a movable platen configured to compress the bladder; and
a drive system configured to move the platen to compress the bladder at a consistent velocity to expel the stored fluid at a substantially steady pressure.

2. The pump of claim 1, wherein the drive system comprises a spring, a decompression of which moves the platen to compress the bladder.

3. The pump of claim 2, wherein the spring is a spiral spring.

4. The pump of claim 3, wherein the platen comprises a shaft extending from a surface opposite the bladder, and wherein the spiral spring is configured to unwind to turn a drive nut affixed to an interior of the housing and having internal threads corresponding to external threads formed on the shaft, the turning of the drive nut causing the shaft to pass therethrough.

5. The pump of claim 4, further comprising a lock slidably engaged over a post extending from the top of the housing, wherein the lock prevents rotation of the drive nut when in a lower position and permits rotation of the drive nut when in an upper position.

6. The pump of claim 5, wherein the lock comprises a plurality of fingers at its lower end configured to engage corresponding slots formed on a top of the drive nut.

7. The pump of claim 1, wherein the drive system comprises one or more magnets on a top of the platen aligned to magnetically oppose corresponding one or more magnets secured on a seat affixed to an interior of the housing, said magnetic opposing moving the platen to compress the bladder.

8. The pump of claim 7, wherein said seat is rotationally coupled to an upper interior surface of the housing, wherein the seat is rotatable to align the corresponding one or more magnets such that they magnetically oppose.

9. The pump of claim 8, wherein the seat is rotatable to unalign the corresponding one or more magnets such that they do not magnetically oppose

10. The pump of claim 9, wherein said substantially steady pressure comprises a range of about 2-4 psi.

11. The pump of claim 10, wherein said substantially steady pressure comprises 2.7 psi.

12. The pump of claim 1, wherein the bladder comprises a fluid conduit for said expelled fluid and having its distal end configured for connection to a cannula sized to engage a grafted human vein.

13. A pump providing a consistent regulated pressurization of an output fluid, the pump comprising:
a housing containing:
a bladder configured to store a fluid therein and comprising a fluid conduit having its distal end configured for connection to a cannula sized to engage a grafted human vein, and
a movable platen having a shaft extending from a first surface and having a second surface, opposite the first surface, configured to compress the bladder; and
a drive system configured to move the platen to compress the bladder at a consistent velocity to expel the stored fluid at a substantially steady pressure, wherein the drive system comprises a spiral spring configured to unwind to turn a drive nut having internal threads corresponding to external threads formed on the shaft, the turning of the drive nut causing the shaft to pass therethrough.

14. The pump of claim 13, further comprising a lock slidably engaged over a post extending from the top of the housing, wherein the lock prevents rotation of the drive nut when in a lower position and permits rotation of the drive nut when in an upper position.

15. The pump of claim 13, wherein said substantially steady pressure comprises a range of about 2-4 psi.

16. The pump of claim 15, wherein said substantially steady pressure comprises 2.7 psi.

17. A pump providing a consistent regulated pressurization of an output fluid, the pump comprising:
a housing containing:
a bladder configured to store a fluid therein and comprising a fluid conduit having its distal end configured for connection to a cannula sized to engage a grafted human vein,
a seat rotatably coupled to an interior surface of the housing, and
a movable platen having an upper surface facing the seat and a lower surface facing the bladder; and
a drive system configured to move the platen to compress the bladder at a consistent velocity to expel the stored fluid at a substantially steady pressure, said drive system comprising:
a plurality of lower magnets secured on an upper surface of the platen opposite the lower surface, and
a plurality of upper magnets secured on the seat, wherein alignment of the upper magnets with corresponding lower magnets results in said magnets magnetically opposing to move the platen to compress the bladder.

18. The pump of claim 17, wherein said seat is rotatable to align the corresponding one or more magnets such that they magnetically oppose, and is further rotatable to unalign the corresponding one or more magnets such that they do not magnetically oppose.

19. The pump of claim 17, wherein said substantially steady pressure comprises a range of about 2-4 psi.

20. The pump of claim 19, wherein said substantially steady pressure comprises 2.7 psi.
